(19) United States
(12) Patent Application Publication
(10) Pub. No.: US 2010/0262149 A1
(43) Pub. Date: Oct. 14, 2010

(54) PROCESS FOR PRODUCING TOOLS USED IN ORTHOPEDIC SURGERIES

(76) Inventors: Dupuy Charles, Montreal (CA); Boisclair Mathieu, Montreal (CA); Julien Benoit, Montreal (CA); Lawson Mark, Montreal (CA)

Correspondence Address: WILMERHALE/BOSTON, 60 STATE STREET, BOSTON, MA, 02109, US

(21) Appl. No.: 12/618,428

(22) Filed: Nov. 13, 2009

Related U.S. Application Data
(60) Provisional application No. 61/114,708, filed on Nov. 14, 2008.

(51) Int. Cl. A61B 17/56; B22F 3/10; B22F 1/00

(52) U.S. Cl. 606/87; 419/44

(57) ABSTRACT

An orthopedic tool being made from an alloy in the group comprising stainless steel alloys, cobalt-chrome alloys, titanium alloys and alumina and zirconia ceramic alloys, and having a density less than 98% of a theoretical possible density for the alloy, the orthopedic tool being made by a metal injection moulding process.

### CROSS REFERENCE TO RELATED APPLICATIONS

This application claims the benefit under 35 USC §119(e) of U.S. provisional patent application Ser. No. 61/114,708 filed Nov. 14, 2008. The contents of the above-mentioned patent application are incorporated herein by reference.

### FIELD OF THE INVENTION

The present invention relates to tools used for orthopedics and arthroplasty, and more specifically to tools used for orthopedics that are formed from a metal injection molding process that imparts certain material characteristics.

### BACKGROUND

Orthopedic tools are known in the art and are commonly made via machining techniques. More specifically, orthopedic tools and tool components are typically machined out of a solid material or are cast followed by machining. This processing is difficult and costly due to the high hardness and poor machinability of adequate materials for the applications.

As such, there is a need in the industry for an improved method and system for producing orthopedic tools that alleviates, at least in part, deficiencies associated with existing methods.

### SUMMARY OF THE INVENTION

In accordance with a first broad aspect, the present invention comprises a surgical cutting guide being made from an alloy in the group comprising stainless steel alloys, cobalt-chrome alloys, titanium alloys and alumina and zirconia ceramic alloys, and having a density less than 98% of a theoretical possible density for the alloy, said surgical cutting guide being made by a metal injection molding process.

In accordance with a second broad aspect, the present invention comprises a process for making a surgical cutting guide, the process comprising:
a. preparing a fluid feedstock including metallic powder selected from the group comprising stainless steel alloys, cobalt-chrome alloys, titanium alloys and alumina and zirconia ceramic alloys and binder material;
b. injecting the feedstock into a mold having cavity approximating the shape of the surgical cutting guide, to form a green part;
c. debinding the green part to provide a debound part;
d. sintering the debound part to yield the surgical cutting guide, wherein the preparing, injecting, debinding and sintering are performed at process conditions such that the surgical cutting guide has a density less than 98% of a theoretical possible density for the alloy.

In accordance with a third broad aspect, the present invention comprises a process for making a surgical cutting guide, the process comprising:
a. preparing a fluid feedstock including metallic powder selected from the group comprising stainless steel alloys, cobalt-chrome alloys, titanium alloys and alumina and zirconia ceramic alloys and binder material;
b. injecting the feedstock into a mold having cavity approximating the shape of the surgical cutting guide, to form a green part;
c. debinding the green part to provide a debound part;
d. sintering the debound part to yield a precursor of the surgical cutting guide, wherein the preparing, injecting, debinding and sintering are performed at process conditions such that the precursor has a density less than 98% of a theoretical possible density for the alloy;
e. performing one or more process step on the precursor to yield the surgical cutting guide, wherein the one or more process steps are such that the surgical cutting guide acquires a higher density than the precursor.

### DESCRIPTION OF DRAWINGS

FIG. 1 shows a non-limiting example of a microstructure.

FIGS. 2-17 show non-limiting examples of tools that can be manufactured according to the present invention.

### DETAILED DESCRIPTION

The present invention relates generally to a method and system for the fabrication of metallic or ceramic tool components and/or tools, some of which are small and have complex shapes, that are used in orthopedic surgeries. In accordance with a non-limiting example, the small complex shaped tools are manufactured via low-pressure injection molding of metallic and/or ceramic powders followed by wick debinding in an alumina wick media and then followed by sintering the material to increase mechanical properties.

The process conditions for the metal injection molding procedure are controlled so as to impart to the orthopedic tools and/or tool components certain material characteristics relating to density, pore sphericity and pore size distribution.

The following is a brief description of the metal injection molding process:

Metal Injection Molding

In accordance with the present invention, each of the orthopedic tools is produced via a metal injection molding process (otherwise known as a powder injection molding process), which gives the tools certain material characteristics relating to density, pore size and pore sphericity. Metal injection molding is a relatively low cost manufacturing process that can produce complex net-shape components from metals, metal alloys, ceramics, cemented carbides and cermets (ceramic-metal composites), among other possibilities.

There are four main steps in a metal injection molding process. The first step is to form a feedstock material by mixing together a powder of the base material/alloy from which the component is to be manufactured, and a binder. The powder can be any fine metallic powder, alloy powder, ceramic powder or carbide powder, depending on the desired material for the final part. As indicated above, some non-limiting examples of alloys that can be used for the orthopedic tools include stainless steel alloys, cobalt-chrome alloys, titanium alloys, alumina and zirconia ceramics.

Although certain examples of alloys are identified above, it should be appreciated that any alloy having desired material characteristics (i.e. mechanical, chemical and physical characteristics) can be used without departing from the spirit of the invention. For example, the alloy from which the part is made may be selected on the basis of desired characteristics relating to oxidation resistance, corrosion resistance and material strength. Corrosion resistance and oxidation resistance can be measured in terms of mm/yr under certain conditions.

The binder that is mixed with the metal alloy powder may be any polymeric binder, and may include a mixture of polymers, such as waxes, dispersants and surfactants. Typical polymers include polyethylene, polyethylene glycol, polymethyl methacrylate, polypropylene. A typical wax that is used is a paraffin wax. It should be appreciated that any type of binder suitable for the intended purpose can be used without departing from the spirit of the invention. The manner in which the appropriate powder and binder are chosen, as well as the percentage of each that is used to form the feedstock, are known to those of skill in the art, and as such will not be described in more detail herein.

During the mixing step, pre-calculated proportions of the powder and binder are mixed together to obtain a homogeneous and predictable feedstock with desirable rheological behaviour. In accordance with a non-limiting example, the powder and binder are hot mixed together using a continuous or batch mixer, and are then cooled and granulated to form the feedstock material to be supplied to an injection molding machine. The powder and binder can be mixed together under isothermal conditions to form a homogenous suspension.

Once the feedstock has been mixed, the process proceeds to the next step during which the feedstock is injected into a suitable mold for being molded into the shape of the desired part. In accordance with a non-limiting embodiment, the feedstock is supplied to the mold using low-pressure injection conditions of less than 100psi. In order to ensure proper filling of the mold under these conditions, the rheological parameters of the feedstock are adjusted in accordance with the molding parameters (i.e. the injection pressure, injection duration, mold material and temperature of the mixture). For example, the feedstock is generally prepared such that it has at least one of its rate of shear, elasticity, plasticity, viscosity and rheological behaviour in relation to temperature and pressure adapted for use with the molding apparatus.

The mold includes a cavity in the shape of the component being formed. In accordance with a non-limiting example, the injection chamber is kept at the same temperature as the mixing chamber, and the injection pressure is typically less than 700 KPa. This pressure is maintained while the part is cooling to prevent void or crack formation due to contraction. The molding time typically takes less than 30 seconds.

The mold can be made from steel, aluminum, bronze, brass or any other metallic material or from polymeric resins such as epoxy, or other thermoplastics, for example. The mold may or may not include another material to facilitate the heat transfer, the shrinkage or any other molding-related aspect. The molds can be hand-made using techniques from the jewelry field or from stereolithography. These mold manufacturing techniques allow reducing development-related and production costs, especially when manufacturing small volumes of components.

The mold is operative for shaping the mixed feedstock into a defined shape, so as to form what is called a “green part”. Once the green part has been formed, meaning that the feedstock has acquired the desired shape and has been removed from the mold, the process proceeds to the debinding step. The purpose of the debinding step is to remove the binder from the powder material, without distorting the molded shape of the green part. Thermal debinding is the most common technique used to debind the part, but any debinding technique can be used without departing from the spirit of the invention. For example, the debinding can be done using solvents, or even water in the case where water soluble polymers, such as polyethylene glycol are used as binders.

In the case of thermal debinding, the molded part is heated in an oven under controlled conditions, such that part of the binder is eliminated at a lower temperature, while the backbone polymer of the binder maintains the powder particles of the molded part in place. This first stage of the debinding process forms a porous network, which eventually helps in the evacuation of the degradation residues from the backbone polymer. It also reduces the internal pressure that could deform the part. The backbone polymer is then thermally removed. Even in the cases where a portion of the binder is removed via solvent, the backbone polymer is generally still thermally removed in a second stage procedure. In some cases, this second stage of the debinding process is performed during the sintering stage, which will be described below, in order to avoid any damage to the debound part.

The final step in the metal injection molding process is the sintering step. During the sintering step the debound part is heated to a temperature that is lower, but close to, the melting temperature of the powder material for bonding the powder particles together. The temperature, duration of heat application and furnace atmosphere are controlled to ensure that the sintered component has the required densification and material properties desired. The sintering step densifies the component by removing the voids left behind from the debinding step. In many cases the sintering step can result in the part shrinking slightly. As such, the mold that is used during the molding stage is designed to compensate for the final shrinkage that occurs during sintering.

Once the metal powder has been processed in the manner described above, the sintered component will have a certain grain size. In accordance with a non-limiting example, the grain size is typically under 75 microns. When using the ASTM E112 standard for grain size characterization, the grain size for static components formed in connection with the present invention are typically, ASTM #4 to ASTM #7. In general, the smaller the grain size, the better the mechanical properties of the end component.

Forming components via metal injection molding can result in cost savings given that there is very little wastage of expensive raw materials. In addition, forming components via metal injection molding can provide increased design and material flexibility, high-speed production and good mechanical properties. In general, the mechanical, physical and chemical properties of the static components formed using the metal injection molding technique described above are comparable to those of wrought material. In addition, components formed from the metal injection molding process require minimal secondary and assembly operations in order to complete the component being manufactured.

Orthopedic Tools and Tool Components

Certain parts/tools for orthopedic use and/or arthroplasty use, formed via a metal injection molding technique according to the present invention, enable the precise positioning, aligning and bracing of a jig on a patient's articulation or bone for a surgical intervention. These parts include cutting guides which are screwed into the bone and present a slot for guiding a saw blade when performing a cut in the bone. Complex shape clamps, broaches, and other orthopedic components can also be formed via a metal injection molding procedure.

These components typically have cross sections ranging from 0.030 inch to ¾ inch and have a weight of less than 300 grams. The parts have features, such as holes, slots and positioning pins, which enable fixation and/or positioning of the parts on a jig which is referenced on the anatomy (fixed on the skeleton), which enables surgical cuts or drilling which is also referenced to the anatomy. Once the intervention (which can be surgical cutting and/or drilling, among other possibilities) is completed, the patient's articulation and/or bone can be fitted with an implant.

The materials of interest for the orthopedic components are stainless steel alloys, cobalt-chrome alloys, titanium alloys and alumina and zirconia ceramics, among other possibilities. For example, other materials can be used if the mechanical, chemical and physical properties provide adequate wear resistance, high yield strength and biocompatibility, for example.

Low Pressure Injection Moulding can enable net shape or near net shape processing of complex shaped parts depending on dimensions and tolerances

The metal injection moulding process is also able to produce components that are consistently able to meet strict tolerance requirements. For example, the tolerance for each dimension can be 0.5% or lower. As such, for a 1 inch dimension, there is a variation of ±0.0025 of an inch. Likewise, for a dimension of ½ an inch, there is a variation of ±0.001 of an inch. In certain circumstances, the metal injection process is able to achieve tolerances lower than 0.3%. In the case of larger parts, in certain circumstances, the tolerances can also be lower than 0.5%.

The metal injection moulding process is also able to produce a set of components from a common injection mold, such that there is a relatively small variation in dimensional tolerance between each component in a given production lot. Each component in the set of components is produced during a different moulding cycle using a common mold. In accordance with a non-limiting example of implementation, the process conditions used during the mixing, injecting, debinding and sintering steps are such that each component in the set of components has a relatively consistent variation in dimensional tolerance. In addition, a consistent variation in dimensional tolerances in components from production lot to production lot is also achieved. In accordance with a non-limiting example, the variation in dimensional tolerance is typically in the range of 0.5%. For components having dimensions in the range of from ¼ to 3″, this process can achieve to results, meaning that approximately 68% of the components in the set of components achieve a dimensional tolerance in the range of 0.5%. These results are even better for components having smaller dimensions. More specifically, for components having dimensions of ⅛″ or less, the metal injection moulding process described above can achieve 6σ results, meaning that 99.9997% of the components have a dimensional tolerance in the range of 0.5%.

In general, the set of components can include anywhere from 200-800 parts, while still maintaining the variation in dimensional tolerances as described above.

In order to measure the dimensional tolerances between components in a production lot, or between components from production lot to production lot, different techniques can be used. Some non-limiting examples of different techniques include CMM testing, and testing using micrometers, callipers and no/no-go gauges. In accordance with a specific non-limiting example, a calliper is used to measure each dimension that has been specified on an engineering drawing. This measurement verification is performed on each part in the production lot.

Processing small batch sizes can be economical since manual and/or soft tooling can be used to inject the parts with low pressure.

The following describes a suitable feedstock and operating parameters for manufacturing an orthopedic component in accordance with the metal injection moulding process described above. In accordance with a specific embodiment, the feedstock includes a powder of gas atomised material (such as a stainless steel alloy, a cobalt-chrome alloy, a titanium alloy, or an alumina and zirconia ceramic) with 80% of the particles having a diameter of less than 22 microns, and a binder that is made of 85% paraffin wax, 5% bees wax, 5% stearic acid and 5% PE-EVA copolymer. The powder and binder are mixed together in proportions suitable for forming a feedstock having between 60-70% solid loading. The powder and binder feedstock is kept at a temperature of 90 C.

Once mixed, the feedstock is injected into a mold that is made out of steel (P20). The mold is kept at a temperature between 25-40 C and preferably between 30-35 C. The feedstock is injected into the mold such that the feedstock is pushed into the mold at a pressure below 60 psi and preferably at a pressure of between 20-40 psi. More specifically, the feedstock is injected into the mold with low pressure such that there is no shearing separation between the powder and the binder as it is being injected. The cycle time used to mold the part is less than 30 seconds.

Once the shaped part is removed from the mold, the debinding process is conducted in a wicking media of high purity alumina powders. More specifically, the parts are buried in the wicking media. The debinding treatment is then conducted under argon gas. The temperature profile applied to the part is as follows: 1) the heat is ramped to 200 C at a rate of 0.5 C/min, and then 2) the heat is ramped from 200 C to 900 C at 0.85 C/min. Once the heat has reached 900 C, it is then held at 900 C for 2 hours, after which time the heat is ramped back down to an ambient temperature.

Once the debinding process is complete, the sintering treatment for a stainless steel 17-4PH is done under pure hydrogen at 1325 C for 1 hour.

Properties of Manufactured Parts

In accordance with a non-limiting example of implementation, materials processed with this technology have relatively high mechanical properties (higher than cast materials, slightly lower than wrought materials). The grain size of the material is less than 75 microns. The density level is greater than 95%.

Sphericity

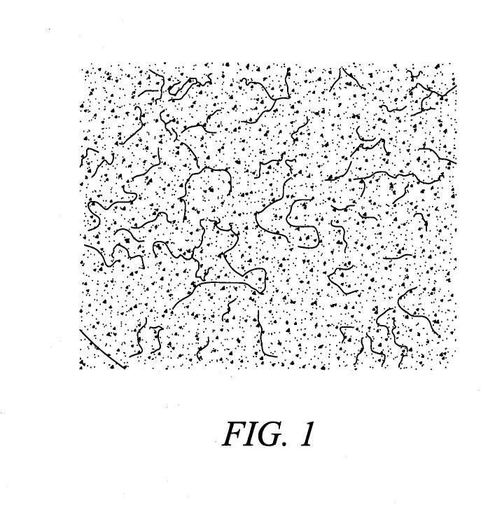

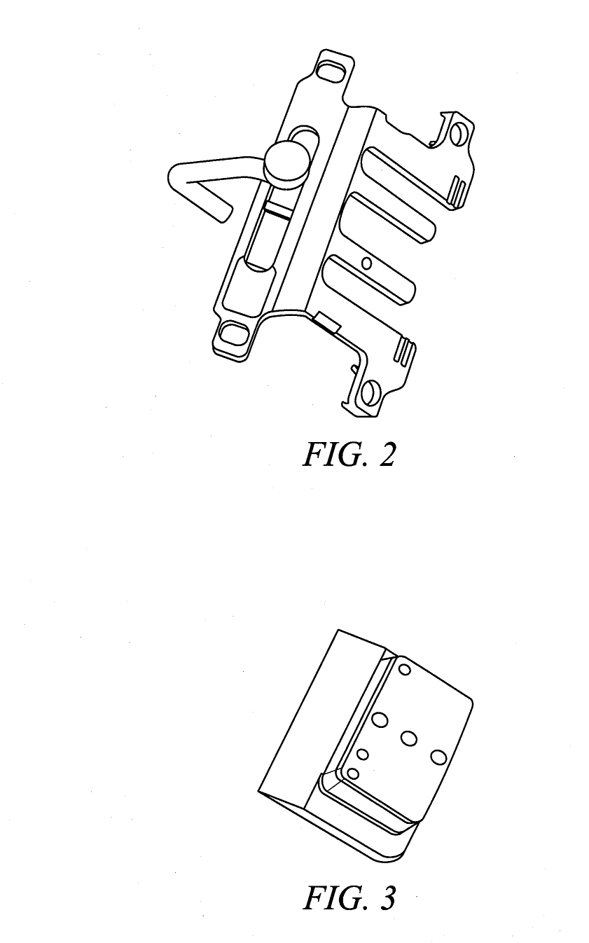

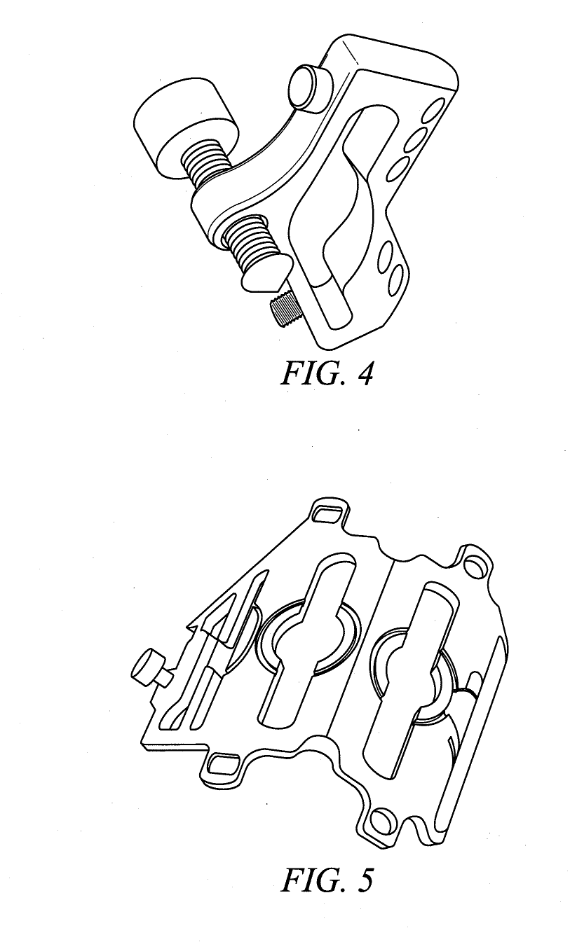

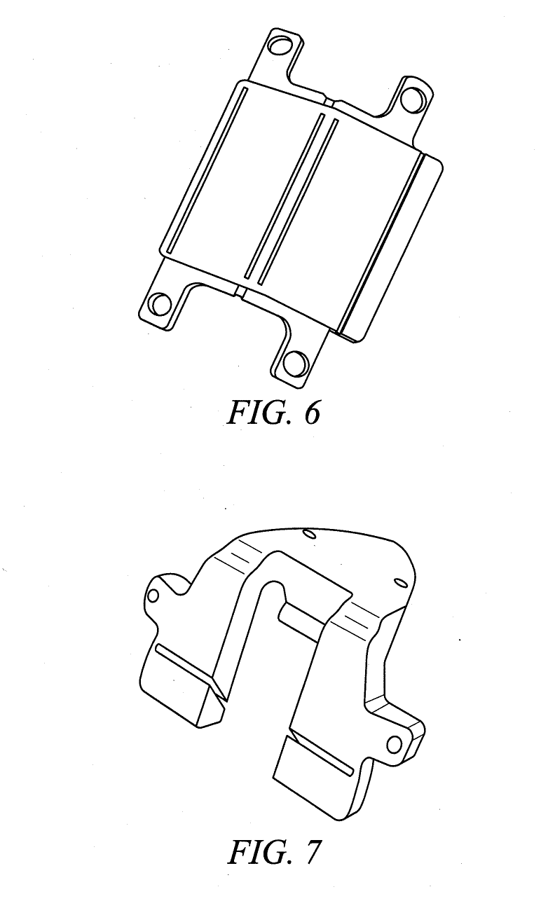

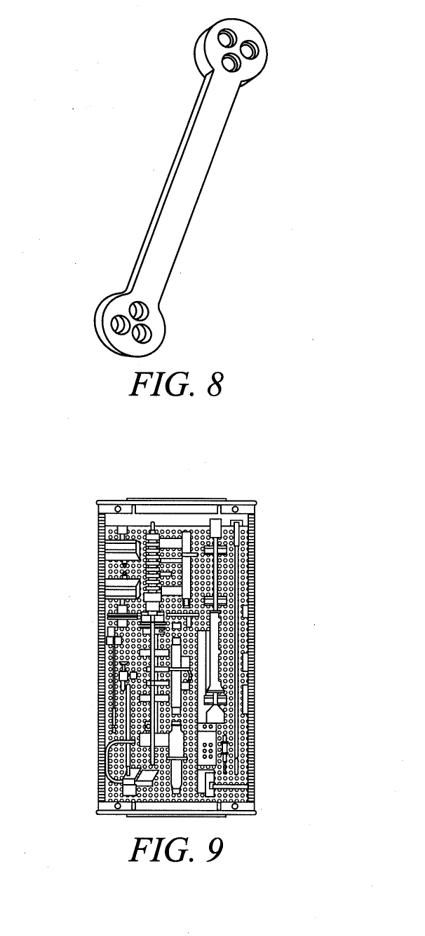

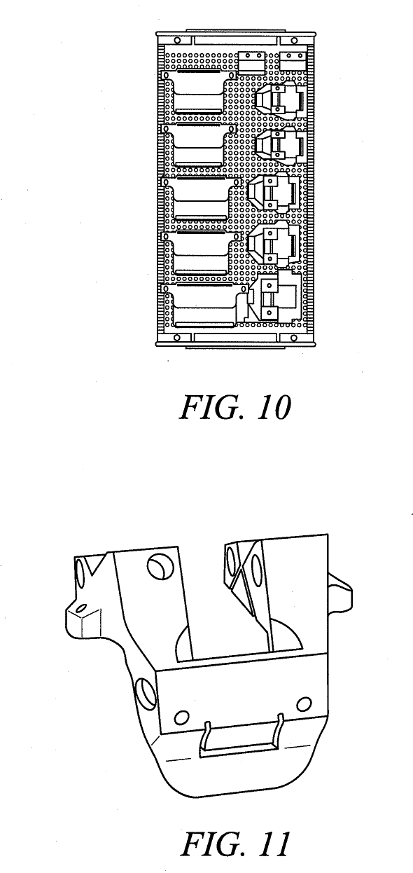

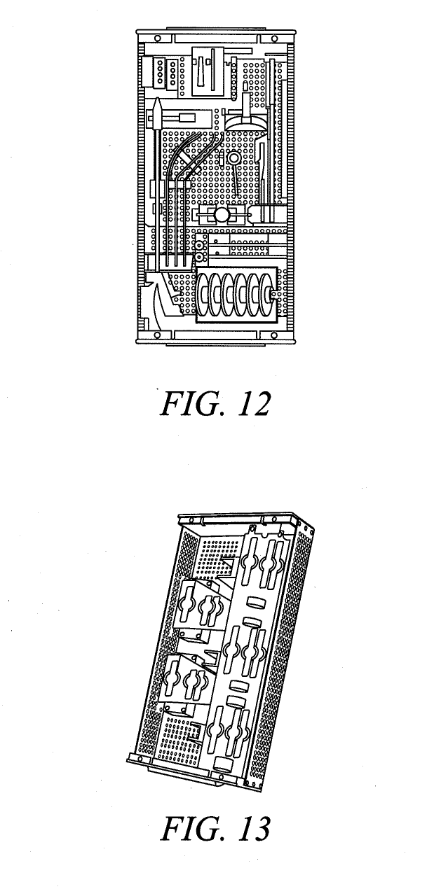

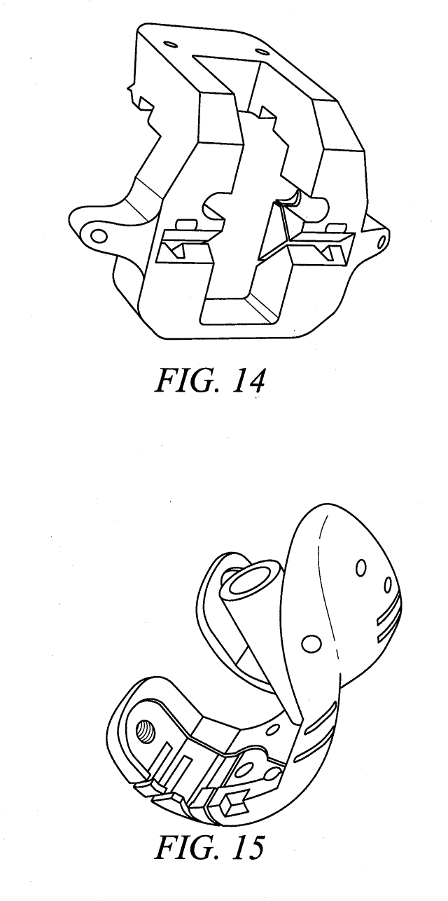

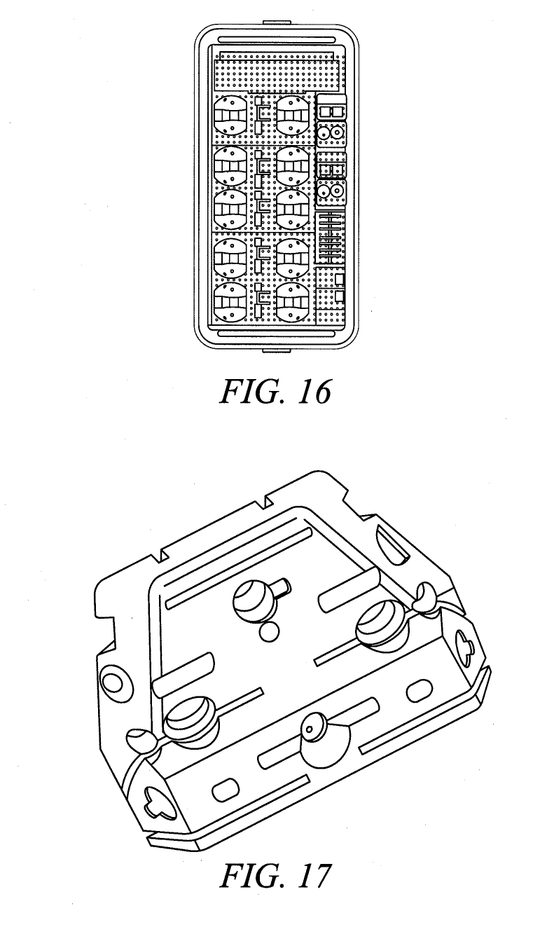

The debinding step that is performed during the metal injection moulding process causes pores to be created in the material from which the orthopedic tools are formed. In this manner, the components produced from the metal injection moulding process are porous components, having a density less than the theoretical density possible in the case where the material does not contain pores. Shown in FIG. 1 is an optical micrograph of a sintered component having pores contained throughout. In the Micrograph of FIG. 1, a combination of pores and second phase particles are shown in black. Ideally, the pores and second phase particles are substantially spherical in shape. The more spherical the pores, the less likely they are to propagate cracks or weakness than if they had a more jagged shape.

In accordance with the present invention, the sphericity of he pores is calculated according to the following formula:

S=(4·π·A)/P²

Where: A=area of the pore
P=perimeter of the pore

The following is a non-limiting example of a method for measuring the sphericity of the pores within an orthopedic tool formed from a low pressure metal injection moulding process. The method involves using a scanning electron microscope, or optical microscope, to capture micrograph images of the microstructure of the component and then analyzing the images using a software program, such as Clémex Vision, to isolate details in the microstructure of the component.

The following is a detailed process used for measuring the sphericity of the pores within a static component:

Step 1—A portion of the component is cut using a slow-cutting saw to expose a cross-sectional (thickness) of the component;

Step 2—A sample of the cut component is prepared for metallographic examination, This preparation involves polishing the sample;

Step 3—Images of the polished sample are captured via a scanning electron microscope. A back scattering technique is used to capture the images. The images are taken at a magnification, which gives a minimum of 150 contrasting features in the image to be analyzed. These contrasting features can be pores or a combination of pores and second phase particles. More specifically, images are taken at magnifications, which enable the analysis of 102-103 contrasting features;

Step 4—The images are imported into Clémex Vision software, and a threshold is created between the contrasting features and the matrix of material. The software then counts the pixels of the contrasting features and transforms the count into dimensions according to a predetermined scale;

Step 5—The imaging software then obtains values in terms of the spherical diameter, sphericity and pore size distribution of the contrasting features. The imaging software is able to use the above formula for calculating the sphericity of the pores. These analyses are done on several images taken in the same conditions on the microscope for a total number of analyzed features greater than 3000;

When processing the contrasting features, it is assumed that the pores and the second phase particles are of roughly the same size.

In accordance with the present invention, the orthopedic tools that are formed from the metal injection moulding process have pores with an average sphericity greater than 0.5. A sphericity of 1 is close to perfect sphericity and a sphericity of 0 is substantially flat. In accordance with a more specific non-limiting embodiment, the pores have an average sphericity greater than 0.7. And in accordance with an even more specific non-limiting embodiment, the pores have an average sphericity of greater than 0.9.

In the case of the micrograph shown in FIG. 1, the software is instructed to provide values for the contrasting features that fall into the following three categories: a) contrasting features larger than 2 μm²; b) contrasting features between 0.5 and 2 μm²; and c) contrasting features smaller than 0.5 μm². The following table outlines the results for this micrograph:

| Microstructural feature based on size | Mean Spherical | |
|---|---|---|
| (μm²) | Diameter (μm) | Sphericity |
| contrasting features larger than 2 μm² | 2.43 | 0.78 |
| contrasting features between 0.5 and | 1.35 | 0.90 |
| 2 μm² | | |
| contrasting features smaller than | 0.50 | 0.99 |
| 0.5 μm² | | |

Pore Size

The pores contained within the orthopedic tools produced from the metal injection moulding procedure of the present invention preferably have a pore size diameter of less than 10 microns. In accordance with a more specific non-limiting example of implementation, the components have pores with an average pore size diameter of less than 5 microns. In accordance with a still more specific non-limiting example of implementation, at least 50% of the pores have a pore size diameter of less than 3 microns.

The above described process that uses a microscope for capturing images of the microstructure of the component and then a software program, such as Clémex Vision, to analyze the captured images can be used in order to obtain the values for the pore size of the pores within the components.

Density

The orthopedic tools formed from the metal injection moulding procedure described above have a density that is less than the theoretical density possible for the material from which the components are made. This is due to the fact that as the binder is removed from the green part, voids are created between the powder particles. These voids turn into substantially spherical pores as the powder particles are thermally bonded together during the sintering process.

As a result, the component, is less dense than the theoretical possible density for the material from which it is made. In other words, the component made from the metal injection process is less dense than if it had been machined from a solid block of the given material. In accordance with a non-limiting example, the static components formed from the above described metal injection moulding process have a density of between 96-99.5% of a theoretical possible density. In accordance with a further non-limiting example, the component has a density of between 97-98% of a theoretical possible density.

The following is a non-limiting example of a method for measuring the density of the components formed from the metal injection moulding process described above. The density of the parts can be evaluated using Archimedes technique, wherein a part is weighed dry and is then weighed again when suspended in water. The difference in weights is due to a buoyant force created by the porosities. This difference in weights enables the calculation of density according to the following equation: SINTERED DENSITY=(dry mass*density of water)/(dry mass−wet mass).

The following is a specific manner in which density is calculated:

Step 1—A sample of the component is taken. The sample can be cut using a slow-cutting saw;

Step 2—The dry sample is weighed using a measuring scale;

Step 3—The sample is then suspended within a body of liquid, and the weight of the suspended sample is taken;

Step 4—The density of the component is determined by entering the dry weight and the weight when suspended in water into the formula DENSITY=(dry mass*density of water)/(dry mass−wet mass). The density can be calculated manually or using a computer program.

Density measurements by the Archimedes technique are ASTM B328 (which is a standard test method for density, oil content and interconnected porosity of sintered metal structural parts) and ASTM B311 of MPIF std. 42.

Surface finish of the sintered materials is typically less than 10 microns (polishing operations and secondary machining can be readily performed on the parts).

1. A surgical cutting guide being made from an alloy in the group comprising stainless steel alloys, cobalt-chrome alloys, titanium alloys and alumina and zirconia ceramic alloys, and having a density less than 98% of a theoretical possible density for the alloy, said surgical cutting guide being made by a metal injection moulding process.

2. A process for making a surgical cutting guide, said process comprising:
a. preparing a fluid feedstock including metallic powder selected from the group comprising stainless steel alloys, cobalt-chrome alloys, titanium alloys and alumina and zirconia ceramic alloys and binder material;
b. injecting the feedstock into a mold having cavity approximating the shape of the surgical cutting guide, to form a green part;
c. debinding the green part to provide a debound part;
d. sintering the debound part to yield said surgical cutting guide, wherein said preparing, injecting, debinding and sintering being performed at process conditions such that said surgical cutting guide has a density less than 98% of a theoretical possible density for the alloy.

3. A process for making a surgical cutting guide, said process comprising:
a. preparing a fluid feedstock including metallic powder selected from the group comprising stainless steel alloys, cobalt-chrome alloys, titanium alloys and alumina and zirconia ceramic alloys and binder material;
b. injecting the feedstock into a mold having cavity approximating the shape of the surgical cutting guide, to form a green part;
c. debinding the green part to provide a debound part;
d. sintering the debound part to yield a precursor of said surgical cutting guide, wherein said preparing, injecting, debinding and sintering being performed at process conditions such that said precursor has a density less than 98% of a theoretical possible density for the alloy;
e. performing one or more process step on said precursor to yield said surgical cutting guide, wherein said one or more process steps are such that said surgical cutting guide acquires a higher density than said precursor.
